(19) United States
(12) Patent Application Publication
(10) Pub. No.: US 2026/0118353 A1
(43) Pub. Date: Apr. 30, 2026

(54) MEASUREMENT DEVICE, OPERATION METHOD OF MEASUREMENT DEVICE, AND OPERATION PROGRAM OF MEASUREMENT DEVICE

(71) Applicant: FUJIFILM CORPORATION, Tokyo (JP)

(72) Inventors: Yuki ARAI, Kanagawa (JP); Kensuke ITO, Kanagawa (JP); Tadahiro MATSUNO, Kanagawa (JP)

(21) Appl. No.: 19/432,096

(22) Filed: Dec. 23, 2025

Related U.S. Application Data
(63) Continuation of application No. PCT/JP2024/021545, filed on Jun. 13, 2024.

(30) Foreign Application Priority Data
Jul. 7, 2023 (JP) 2023-112651

(51) Int. Cl. G01N 33/543; G01N 21/64; G01N 33/58

(52) U.S. Cl. CPC G01N 33/54373; G01N 21/6428; G01N 33/582; G01N 2021/6439

(57) ABSTRACT

A measurement device is configured to use an analysis chip having a flow path through which a specimen solution containing a target substance flows, the flow path being provided with a reaction region in which an antibody or an antigen that specifically reacts with the target substance is immobilized and with which the specimen solution comes into contact, and to measure a reaction of the antibody or the antigen with the target substance in the reaction region while feeding the specimen solution, the measurement device including a processor, in which the processor is configured to acquire flow rate-related information related to a flow rate of the specimen solution in the flow path, and to execute post-processing related to reliability of a measurement result based on the flow rate-related information.

### CROSS-REFERENCE TO RELATED APPLICATIONS

This application is a continuation application of International Application No. PCT/JP2024/021545, filed June 13, 2024, the disclosure of which is incorporated herein by reference in its entirety. Further, this application claims priority from Japanese Patent Application No. 2023-112651, filed on July 7, 2023, the disclosure of which is incorporated herein by reference in its entirety.

### BACKGROUND

### 1. Technical Field

The present disclosure relates to a measurement device, an operation method of a measurement device, and an operation program of a measurement device.

### 2. Description of the Related Art

As in the fluorescence detection device using surface plasmon described in JP5640980B, a measurement device configured to use an analysis chip having a flow path through which a specimen solution containing a test substance flows, and to measure an antigen-antibody reaction in a reaction region while feeding the specimen solution is known. The test substance is an antigen or an antibody, and a binding substance to be immobilized in the reaction region changes depending on whether the test substance is an antigen or an antibody. For example, in a case where the test substance is an antigen, an antibody is immobilized in the reaction region, and in a case where the test substance is an antibody, an antigen is immobilized in the reaction region.

### SUMMARY

A method of measuring the reaction while feeding the specimen solution is called a liquid feeding method or the like. The liquid feeding method tends to reduce the likelihood that a bias in the concentration of the test substance occurs in the specimen solution, as compared with a method of measuring the reaction in a state where the specimen solution is allowed to remain, and tends to accurately detect the concentration of the test substance. The feeding of liquid is performed by applying a constant liquid feeding pressure to the specimen solution.

However, even in a case where the liquid feeding pressure of the specimen solution is kept constant, there are individual differences in the viscosity of specimen solutions, and there are cases where the target flow rate may not be reached. In a case where the flow rate does not reach the target flow rate, a bias in the concentration of the test substance occurs in the specimen solution, and there is a possibility that the concentration of the test substance cannot be accurately detected. Since it is not preferable that such a measurement result with concern about reliability is directly presented to a user, some measures have been desired.

One embodiment according to the technology of the present disclosure provides a measurement device, an operation method of a measurement device, and an operation program of a measurement device, which makes is possible to take an appropriate measure against reliability of a measurement result even in a case where there are individual differences in viscosity of specimen solutions.

A measurement device according to the technology of the present disclosure is configured to use an analysis chip having a flow path through which a specimen solution containing a target substance flows, the flow path being provided with a reaction region in which an antibody or an antigen that specifically reacts with the target substance is immobilized and with which the specimen solution comes into contact, and to measure a reaction of the antibody or the antigen with the target substance in the reaction region while feeding the specimen solution, the measurement device including a processor, in which the processor is configured to acquire flow rate-related information related to a flow rate of the specimen solution in the flow path, and to execute post-processing related to reliability of a measurement result based on the flow rate-related information.

It is preferable that the flow rate-related information is an arrival time until a head of the specimen solution arrives at a predetermined position in the flow path after initiation of the feeding of the specimen solution.

It is preferable that in a case where the arrival time exceeds a predetermined first threshold value, the processor is configured to add supplementary information related to reliability to the measurement result as the post-processing.

It is preferable that in a case where the arrival time exceeds a predetermined first threshold value, the processor is configured to correct the measurement result as the post-processing.

It is preferable that the liquid feeding pressure of the specimen solution is variable, and in a case where a plurality of the specimen solutions are consecutively measured and the plurality of the specimen solutions that are consecutively measured are generated from the same specimen, the processor is configured, in a case where the arrival time exceeds a predetermined first threshold value in the measurement of one specimen solution, to increase the liquid feeding pressure in a case of measuring another specimen solution that is consecutively measured as the post-processing.

It is preferable that the specimen solution contains a label substance that is bindable to the test substance, a capture region that captures the label substance regardless of whether or not the label substance is bonded to the test substance is disposed on a downstream side of the reaction region of the flow path, and the flow rate-related information is a concentration of the label substance in the capture region or the flow rate of the specimen solution derived based on the concentration of the label substance.

It is preferable that in a case where the concentration of the label substance or the flow rate is equal to or less than a predetermined second threshold value, the processor is configured to add supplementary information related to reliability to the measurement result as the post-processing.

It is preferable that in a case where the concentration of the label substance or the flow rate is equal to or less than a predetermined second threshold value, the processor is configured to correct the measurement result as the post-processing.

It is preferable that the liquid feeding pressure of the specimen solution is variable, and in a case where a plurality of the specimen solutions are consecutively measured and the plurality of the specimen solutions that are continuously measured are generated from the same specimen, the processor is configured, in a case where the concentration of the label substance or the flow rate is equal to or less than a predetermined second threshold value in the measurement of one specimen solution, to increase the liquid feeding pressure in a case of measuring another specimen solution that is consecutively measured as the post-processing.

It is preferable that the reaction is measured by using surface plasmon resonance.

An operation method of a measurement device according to the technology of the present disclosure is an operation method of a measurement device that is configured to use an analysis chip having a flow path through which a specimen solution containing a target substance flows, the flow path being provided with a reaction region in which an antibody or an antigen that specifically reacts with the target substance is immobilized and with which the specimen solution comes into contact, and to measure a reaction of the antibody or the antigen with the target substance in the reaction region while feeding the specimen solution, the operation method including executing, by a processor, processes including: a process of acquiring flow rate-related information related to a flow rate of the specimen solution in the flow path; and a process of executing post-processing related to reliability of a measurement result based on the flow rate-related information.

An operation program of a measurement device according to the technology of the present disclosure is an operation program of a measurement device that is configured to use an analysis chip having a flow path through which a specimen solution containing a target substance flows, the flow path being provided with a reaction region in which an antibody or an antigen that specifically reacts with the target substance is immobilized and with which the specimen solution comes into contact, and to measure a reaction of the antibody or the antigen with the target substance in the reaction region while feeding the specimen solution, the operation program causing a computer to execute processes including: a process of acquiring flow rate-related information related to a flow rate of the specimen solution in the flow path; and a process of executing post-processing related to reliability of a measurement result based on the flow rate-related information.

According to the present disclosure, an appropriate measure against reliability of a measurement result can be taken even in a case where there are individual differences in viscosity of specimen solutions.

### BRIEF DESCRIPTION OF THE DRAWINGS

FIG. 1 is an external view of a measurement device according to the present disclosure.

FIG. 2 is a block diagram of the measurement device.

FIG. 3 is a schematic diagram showing an example of an analysis chip used in the measurement device.

FIG. 4 is a schematic diagram showing a state in which a specimen is extracted from a specimen container by using a nozzle tip by a specimen processing unit.

FIG. 5 is a schematic diagram showing a state in which the specimen in a nozzle tip is injected and stirred in a reagent cell by the specimen processing unit.

FIG. 6 is a diagram showing a positional relationship between a flow path and a measurement unit and a moving method of the measurement unit.

FIG. 7 is a diagram illustrating an outline of fluorescence detection using the measurement unit.

FIG. 8 is a diagram showing an incidence angle of excitation light and a plasmon enhancement factor.

FIG. 9 is a diagram showing a configuration for detecting a head of the specimen solution flowing in the flow path.

FIG. 10 is a diagram showing a concentration distribution of the test substance in the flow path in a case where the flow rate is high.

FIG. 11 is a diagram showing a concentration distribution of the test substance in the flow path in a case where the flow rate is low.

FIG. 12 is a graph showing a relationship between the viscosity of the specimen solution and the flow rate.

FIG. 13 is an explanatory diagram of an arrival time.

FIG. 14 is a diagram showing a process of adding supplementary information to a measurement result.

FIG. 15 is a flowchart showing an operation procedure of the measurement device.

FIG. 16 is a diagram showing a process of correcting the measurement result.

FIG. 17 is a diagram showing a process of increasing a liquid feeding pressure in a case of next measurement.

FIG. 18 is a graph showing a relationship between the flow rate of the specimen solution and a measured value of a concentration of a fluorescent label.

FIG. 19 is a diagram showing a process of adding supplementary information to a measurement result in a second embodiment.

### DETAILED DESCRIPTION

### First embodiment

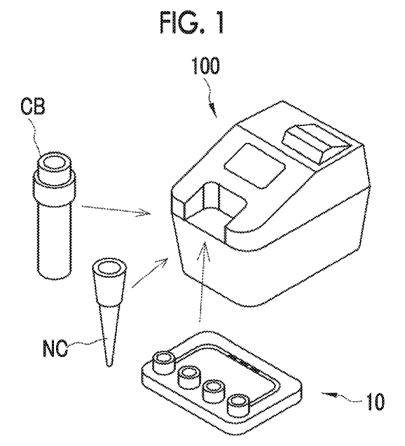

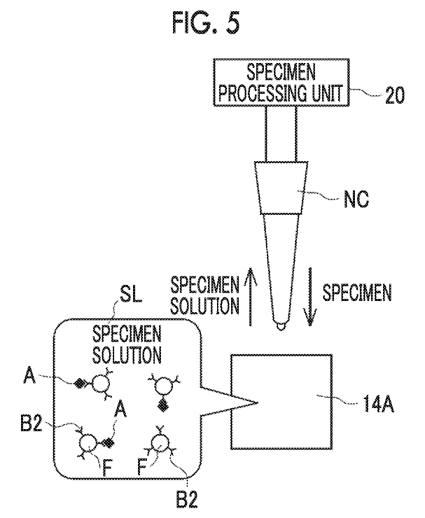

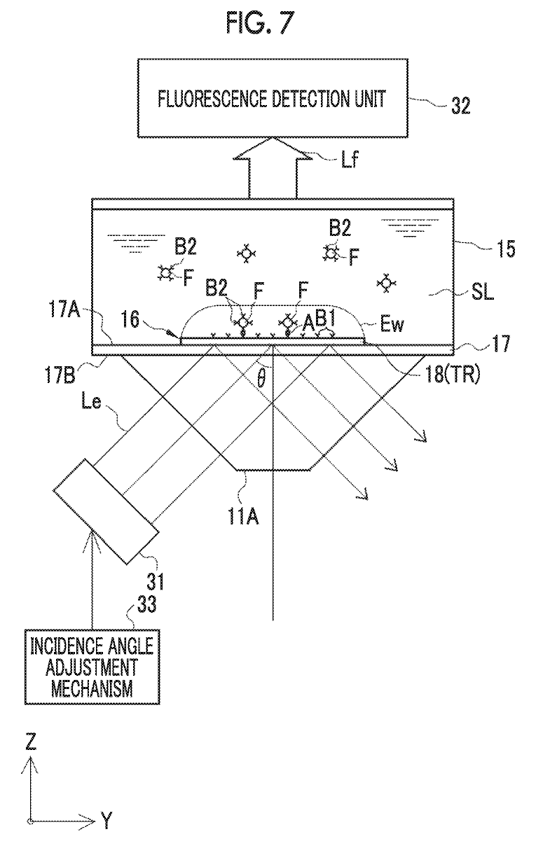

A measurement device 100 shown in FIG. 1 is, as an example, a measurement device that measures an antigen-antibody reaction of a test substance A (see FIG. 4 and the like) contained in a specimen collected from a living body for performing immunodiagnosis. The measurement device 100 is, as an example, a measurement device using a fluorescence method. The fluorescence method is a measurement method of measuring the antigen-antibody reaction of the test substance A by irradiating a fluorescent label F (see FIG. 5, FIG. 7, and the like) bonded to the test substance A with excitation light and detecting fluorescence generated from

the fluorescent label F. More specifically, the measurement device 100 measures the antigen-antibody reaction of the test substance A by enhancing the fluorescence emitted from the fluorescent label F by using surface plasmon resonance phenomenon. Such a measurement method is called surface plasmon field-enhanced fluorescence spectroscopy (SPFS) or the like.

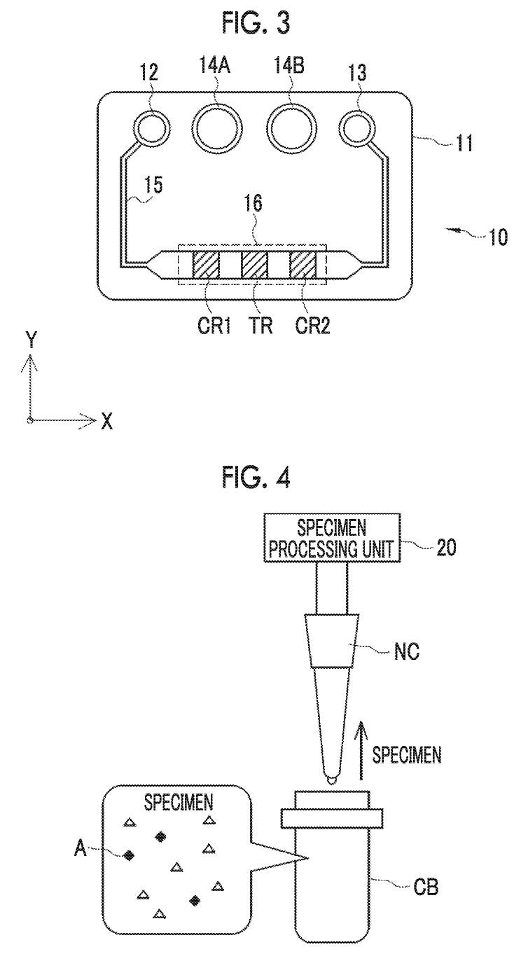

In a case of performing the measurement using the measurement device 100, a specimen container CB in which the specimen shown in FIG. 1 is accommodated, a nozzle tip NC used in a case of extracting the specimen and a reagent, and an analysis chip 10 on which a reagent cell and a microchannel are formed are set in the measurement device 100. It is noted that the specimen container CB, the nozzle tip NC, and the analysis chip 10 are all disposable items that are discarded after being used once. Then, the measurement device 100 injects the specimen into a flow path 15 (see FIG. 3 and the like) of the analysis chip 10, and performs quantitative measurement of the test substance A in the specimen, as an example.

The specimen is, for example, blood, and more specifically, serum, blood plasma, or whole blood. It is noted that the specimen may be other than blood, and may be urine, nasal fluid, saliva, stool, body cavity fluid, or the like. The test substance A contained in the specimen is, for example, a nucleic acid, a protein, an amino acid, a sugar, a lipid, a modified molecule thereof, a complex, or the like. The complex may be, for example, a tumor marker, a signal transduction substance, a hormone, or the like.

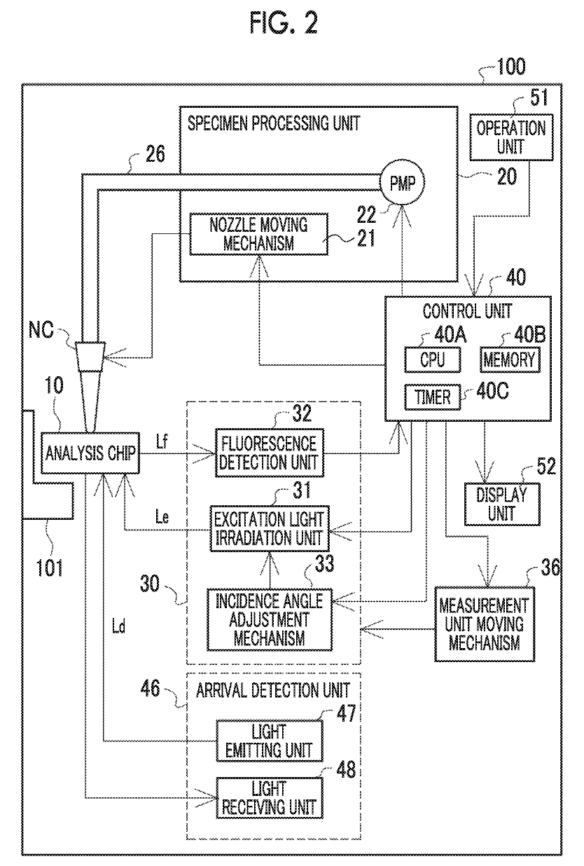

As shown in FIG. 2, the measurement device 100 includes a mounting portion 101, a specimen processing unit 20, a measurement unit 30, a control unit 40, and the like. The analysis chip 10 is mounted on the mounting portion 101. The specimen processing unit 20 extracts the specimen from the specimen container CB (see FIG. 1) by using the nozzle tip NC, and generates a specimen solution SL (see FIG. 5 and the like) in which the extracted specimen is mixed and stirred with a reagent. In addition, the specimen processing unit 20 injects the generated specimen solution SL into the analysis chip 10.

Specifically, the specimen processing unit 20 includes a nozzle moving mechanism 21, a pump 22, and the like. The nozzle moving mechanism 21 is a mechanism for moving the nozzle tip NC in an up-down direction and a left-right direction. The pump 22 is connected to the nozzle tip NC via a pipe 26, and performs delivery and suction of a liquid such as the specimen through a gas.

The measurement unit 30 measures the reaction of the test substance A contained in the specimen solution SL injected into the analysis chip 10 by using the fluorescence method using the surface plasmon resonance. The measurement unit 30 includes an excitation light irradiation unit 31, an incidence angle adjustment mechanism 33, a fluorescence detection unit 32, and the like.

The excitation light irradiation unit 31 irradiates the analysis chip 10 with excitation light Le. The excitation light irradiation unit 31 is composed of, for example, a laser diode (LD) as light emitting unit that emits the excitation light Le, a reflection mirror that reflects the excitation light Le, and the like. The incidence angle adjustment mechanism 33 adjusts an incidence angle of the excitation light Le to be irradiated to the analysis chip 10. The fluorescence detection unit 32 detects the fluorescence emitted from the fluorescent label F excited by the excitation light Le in the analysis chip 10, and outputs a fluorescence detection signal to the control unit 40. The fluorescence detection unit 32 is composed of a photodiode, a photomultiplier, a charge coupled device (CCD) image sensor, a complementary metal oxide semiconductor (CMOS) image sensor, and the like.

The measurement unit moving mechanism 36 is a moving mechanism that moves the measurement unit 30. As will be described later, a plurality of regions to be measured are provided in the analysis chip 10, and the measurement unit moving mechanism 36 moves the measurement unit 30 with respect to the analysis chip 10 such that the measurement of the plurality of regions of the analysis chip 10 can be performed.

The control unit 40 integrally controls each unit of the measurement device 100. An operation unit 51 and a display unit 52 are connected to the control unit 40. In addition, the control unit 40 has a built-in timer 40C that performs various types of timing. The operation unit 51 is composed of a button, a cross key, and the like, and inputs an operation instruction such as a measurement start instruction to the control unit 40. In addition, input of patient information related to the specimen and the like is also performed through the operation unit 51. The display unit 52 is composed of, for example, a liquid crystal panel, and the like, and displays a measurement result, a status indicating an operation state, a message such as a warning.

In response to the measurement start instruction from the operation unit 51, the control unit 40 controls the specimen processing unit 20 to inject the specimen solution SL into the analysis chip 10. Then, the measurement is performed by operating the measurement unit moving mechanism 36 and the measurement unit 30. In the measurement, the control unit 40 outputs, as the measurement result, a concentration of the test substance A, as an example, based on the fluorescence detection signal acquired from the fluorescence detection unit 32. It is noted that data analysis may be performed based on the concentration, and the analysis result may be output together with the measurement result, in addition to the concentration of the test substance A. The control unit 40 outputs the measurement result to the display unit 52.

The control unit 40 includes, as an example, a central processing unit (CPU) 40A, a memory 40B, and the timer 40C. In addition, the control unit 40 is communicably connected to a data storage not shown (not illustrated). As is well known, the CPU 40A executes processing defined in a program by executing the program loaded in the memory 40B. The memory 40B includes a random access memory (RAM) and a read only memory (ROM). The data storage is a hard disk drive (HDD), a solid state drive (SSD), or the like. The control unit 40 including the CPU 40A is an example of a processor according to the technology of the present disclosure. The program is an operation program for causing the CPU 40A, which is an example of a computer, to function as the measurement device. The operation program is stored in, for example, the memory 40B or the data storage.

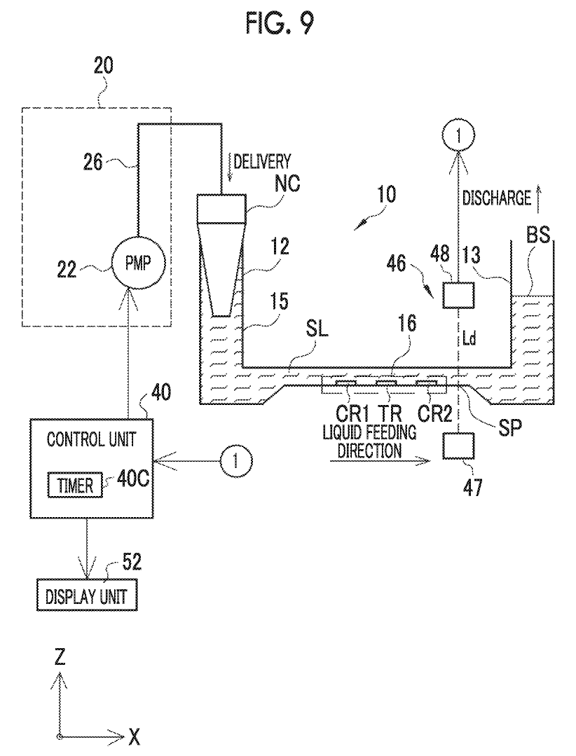

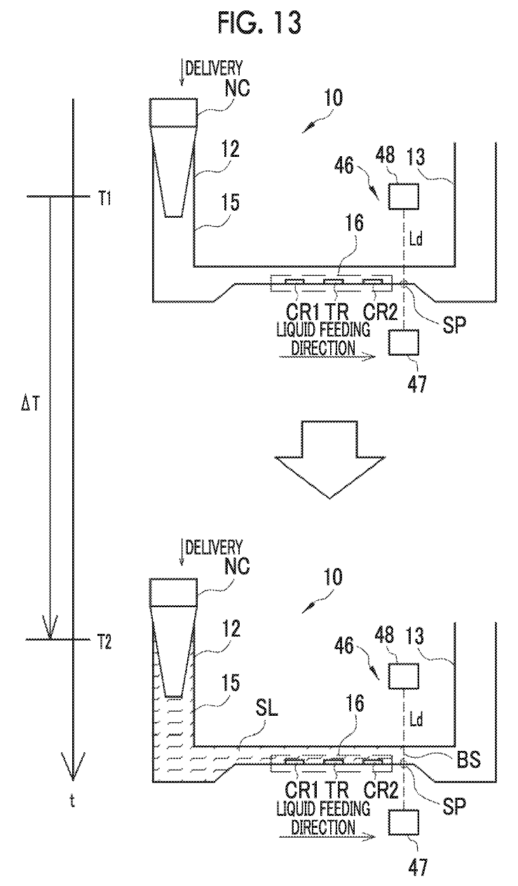

In a case where the specimen solution SL is injected into the flow path 15, the arrival detection unit 46 detects whether or not a head BS (see FIG. 9 and FIG. 13) of the specimen solution SL has arrived at a set position SP (see FIG. 9) set in advance in the flow path 15. The arrival detection unit 46 is composed of, as an example, a light emitting unit 47 that emits detection light Ld and a light receiving unit 48 that receives the detection light Ld, and optically detects the arrival of the specimen solution SL. The light emitting unit 47 is, as an example, an LED, and the light receiving unit 48 is, as an example, a photodiode. The light emitting unit 47 and the light receiving unit 48 are disposed at positions facing each other across the flow path 15. The light receiving unit 48 receives the detection light Ld transmitted through the flow path 15. A received light amount of the detection light Ld received by the light receiving unit 48 changes depending on the presence or absence of the specimen solution SL at a transmission position of the detection light Ld. The arrival detection unit 46 outputs a light receiving signal corresponding to the light amount received by the light receiving unit 48 to the control unit 40.

The control unit 40 detects the arrival of the specimen solution SL based on the light receiving signal from the arrival detection unit 46. As a result, a switching timing between primary liquid feeding and secondary liquid feeding of the specimen solution SL, which will be described later, is detected. In addition, the control unit 40 measures an arrival time ΔT until the head BS of the specimen solution SL arrives at the set position SP in the flow path 15 after the feeding of the specimen solution SL into the flow path 15 is started, by using the arrival detection unit 46 and the timer 40C. The arrival time ΔT is an example of flow rate-related information related to the flow rate of the specimen solution SL in the flow path 15, as will be described later.

FIG. 3 is a schematic diagram showing an example of the analysis chip 10. The analysis chip 10 has a structure in which an injection port 12, a discharge port 13, reagent cells 14A and 14B, and a flow path 15 are formed on a main body 11 formed of a dielectric such as a light-transmitting resin. The injection port 12 communicates with the discharge port 13 via the flow path 15. The nozzle tip NC is inserted into the injection port 12. The specimen processing unit 20 injects the specimen solution SL into the flow path 15 via the nozzle tip NC. The reagent cells 14A and 14B are containers that accommodate a fluorescent reagent to be mixed with the specimen contained in the specimen container CB. The fluorescent reagent is subjected to pretreatment such as adsorbing to a protein in the specimen to deviate a target for pH adjustment. It is noted that opening portions of the reagent cells 14A and 14B are sealed with a sealing member, and the sealing member is configured to be perforated when the specimen is mixed with the fluorescent reagent.

In addition, a reaction region 16 for detecting the test substance A in the specimen is provided in the flow path 15. A test region TR and first control region CR1 and second control region CR2 are formed in the reaction region 16. In the flow path 15, in a case where a side on which the injection port 12 is provided is defined as an upstream side of the reaction region 16, the first control region CR1, the test region TR, and the second control region CR2 are provided in this order from the upstream side to a downstream side.

A first antibody B1 is immobilized on the test region TR, and the test substance A is captured. The first antibody B1 is an example of an antibody that specifically reacts with the test substance A. In addition, the first control region CR1 is a region that normally does not capture anything, and is a so-called negative-type control region in which a signal value serving as a base of the fluorescence detection signal is 0. The second control region CR2 is a region in which a substance that captures the fluorescent label F in the specimen solution SL is immobilized. The second control region CR2 captures the fluorescent label F regardless of whether or not the test substance A is bonded thereto. Therefore, the second control region CR2 is a region in which a signal value serving as a base of the fluorescence detection signal is a value corresponding to the concentration of the fluorescent label F contained in the specimen solution SL, and is a so-called positive-type control region. For example, specimen abnormality, measurement abnormality, and the like are detected based on the fluorescence detection signals of the first control region CR1 and the second control region CR2.

Then, in a case where the measurement start is instructed, the specimen processing unit 20 suctions the specimen from the specimen container CB by using the nozzle tip NC as shown in FIG. 4. Thereafter, as shown in FIG. 5, the specimen processing unit 20 perforates the sealing member of the reagent cell 14A, mixes and stirs the specimen with the reagent in the reagent cell 14A, and then again suctions the specimen solution SL again by using the nozzle tip NC. The same operation is performed on the reagent cell 14B. The reagent is a second antibody B2 labeled with the fluorescent label F. The second antibody B2 specifically binds to the test substance A present in the specimen. Therefore, by mixing and stirring the specimen with the reagent, the specimen solution SL is generated in which the second antibody B2 and the fluorescent label F are modified on a surface of the test substance A by bonding between the second antibody B2 and the test substance A.

Then, the specimen processing unit 20 inserts the nozzle tip NC accommodating the specimen solution SL into the injection port 12. Then, by operating the pump 22 to perform a discharge operation of discharging the specimen solution SL from the nozzle tip NC, the specimen solution SL in the nozzle tip NC is injected into the flow path 15. In synchronization with the discharge operation from the injection port 12, the atmosphere may be suctioned from the outlet port 13. In this manner, the specimen solution SL can be smoothly injected into the flow path 15.

In a case where the injection of the specimen solution SL is started by the discharge operation of the pump 22, the feeding of the specimen solution SL in the flow path 15 is started. A discharge pressure of the pump 22 is a liquid feeding pressure for feeding the specimen solution SL. The control unit 40 can change the liquid feeding pressure of the specimen solution SL by changing the discharge pressure of the pump 22. The control unit 40 controls the liquid feeding pressure of the specimen solution SL by controlling the discharge pressure of the pump 22.

The control unit 40 changes the liquid feeding pressure in the primary liquid feeding and the secondary liquid feeding. The primary liquid feeding refers to feeding of liquid until the specimen solution SL arrives to a set position SP (see FIG. 9) set in advance in the flow path 15 after the feeding of the specimen solution SL is started, and the secondary liquid feeding refers to feeding of liquid during which the measurement unit 30 starts operation and the fluorescence detection as the measurement by the measurement unit 30 is performed after the primary liquid feeding is ended.

The liquid feeding pressure of the primary liquid feeding is set to be higher than the liquid feeding pressure of the secondary liquid feeding. The control unit 40 relatively increases the flow rate of the specimen solution SL in the primary liquid feeding by relatively increasing the liquid feeding pressure of the primary liquid feeding. As a result, the primary liquid feeding can be ended in a short time. On the other hand, the control unit 40 decreases the liquid feeding pressure in the secondary liquid feeding to relatively decrease the flow rate of the specimen solution SL. As a result, the accuracy of the measurement (that is, the fluorescence detection) by the measurement unit 30 is improved. The flow rate of the primary liquid feeding is, for example, about 100 μl/min to 200 μl/min, and the flow rate of the secondary liquid feeding is, for example, about 10 μl/min to 20 μl/min, which is about one tenth of the flow rate of the primary liquid feeding. As a result, the measurement time of the measurement unit 30 is secured for about several minutes.

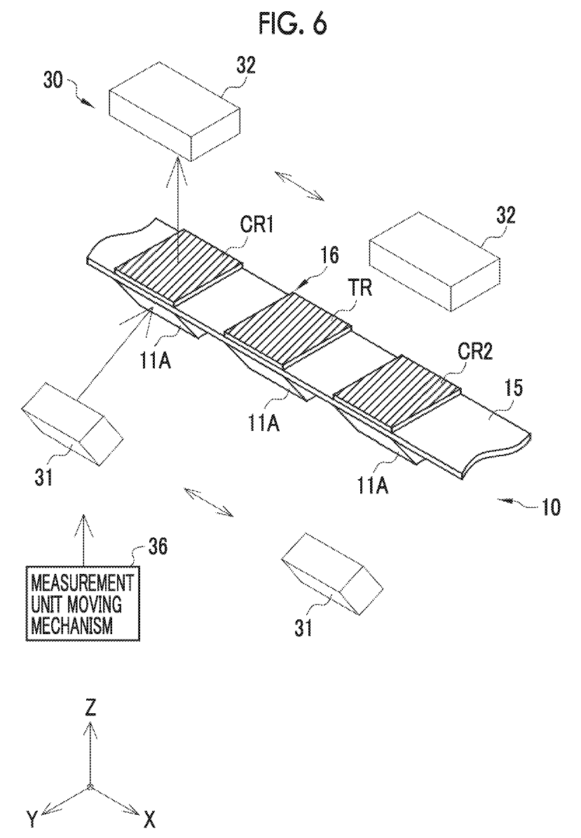

FIG. 6 is an explanatory diagram showing the first control region CR1, the test region TR, and the second control region CR2 of the analysis chip 10, and a state in which the measurement unit 30 moves with respect to the analysis chip 10. The first control region CR1, the test region TR, and the second control region CR2 are disposed along a flow direction (X direction) of the specimen solution SL in the flow path 15. A prism 11A having an incidence surface on which the excitation light Le is incident is provided in the main body 11 corresponding to each region of the first control region CR1, the test region TR, and the second control region CR2.

In the measurement unit 30, the excitation light irradiation unit 31 is disposed at a position facing the incidence surface of the prism 11A of the analysis chip 10 in a case where the analysis chip 10 is mounted on the mounting portion 101. On the other hand, the fluorescence detection unit 32 is disposed at a position facing the first control region CR1, the test region TR, and the second control region CR2 above the flow path 15 of the analysis chip 10, and is disposed at a position where fluorescence from each region can be detected.

The measurement unit moving mechanism 36 linearly moves the excitation light irradiation unit 31 and the fluorescence detection unit 32 along the flow direction (X direction) of the flow path 15, that is, the arrangement direction of the first control region CR1, the test region TR, and the second control region CR2. As a result, the measurement unit 30 can selectively move to a position facing each region of the first control region CR1, the test region TR, and the second control region CR2 to measure the reaction of each region.

FIG. 7 is an explanatory diagram showing a relationship between the reaction region 16 of the analysis chip 10 and the excitation light irradiation unit 31 and the fluorescence detection unit 32 as viewed from the X direction. It is noted that, in FIG. 7, the test region TR is focused on for description, but the same applies to the first control region CR1 and the second control region CR2.

The main body 11 of the analysis chip 10 includes a dielectric plate 17. A front surface 17A of the dielectric plate 17 constitutes a bottom surface of the flow path 15, and the prism 11A is provided on a back surface 17B. A metal film 18 constituting the test region TR, the first control region CR1, and the second control region CR2 is formed on the dielectric plate 17. A material of the metal film 18 is gold in the present example. The dielectric plate 17 and the prism 11A are integrally molded, and the prism 11A is also a dielectric.

In the dielectric plate 17, the front surface 17A corresponds to a main surface that is in contact with a back surface of the metal film 18 opposite to a surface on which the test region TR corresponding to the reaction region is provided.

As described above, the first antibody B1 is immobilized on the metal film 18 of the test region TR, and the first antibody B1 captures the test substance A modified with the fluorescent label F and the second antibody B2 by a so-called sandwich method. As described above, the first control region CR1 is a negative-type control region, and, for example, no antibody is immobilized on the metal film 18 of the first control region CR1. That is, the first control region CR1 is merely the metal film 18. In addition, as described above, the second control region CR2 is a positive-type control region, and a substance that captures the fluorescent label F regardless of the presence or absence of the test substance A is immobilized on the metal film 18 of the second control region CR2.

The excitation light irradiation unit 31 causes the excitation light Le to be incident on the surface 17A from a back side of the front surface 17A of the dielectric plate 17 in contact with the back surface of the metal film 18, via the prism 11A. An incidence angle θ of an optical axis with respect to the surface 17A is an angle equal to or larger than a critical angle satisfying a total reflection condition. As a result, the excitation light Le is irradiated to the back surface of the metal film 18 of the test region TR, the first control region CR1, and the

second control region CR2. As described above, a reflection mirror is provided in the excitation light irradiation unit 31. The reflection mirror can be rotationally moved, and the excitation light irradiation unit 31 can change the incidence angle θ of the excitation light Le by rotationally moving the reflection mirror. The incidence angle adjustment mechanism 33 adjusts the incidence angle of the excitation light Le by rotationally moving the reflection mirror by using a lens or the like, without changing the irradiation position of the excitation light Le on the back surface of the metal film 18.

In a case where the excitation light Le is incident on the back surface of the metal film 18 at a specific incidence angle equal to or larger than the critical angle by the excitation light irradiation unit 31, an evanescent wave Ew extends on the metal film 18, and the surface plasmon are excited on a surface of the metal film 18 by the evanescent wave Ew. The surface plasmon causes an electric field distribution to occur on the surface of the metal film 18, and an electric field enhancement region is formed. Then, the fluorescent label F bonded to the first antibody B1 immobilized on the metal film 18 generates enhanced fluorescence Lf by being excited by the evanescent wave Ew. The fluorescence detection unit 32 receives the enhanced fluorescence Lf and outputs a fluorescence detection signal corresponding to a light amount of the received fluorescence Lf.

Here, the specific incidence angle θ at which the fluorescence Lf enhanced by occurrence of surface plasmon resonance is maximized is referred to as a resonance angle. The resonance angle changes depending on a type of the specimen solution SL in contact with the surface of the metal film 18. Therefore, the incidence angle θ of the excitation light Le is adjusted by the incidence angle adjustment mechanism 33.

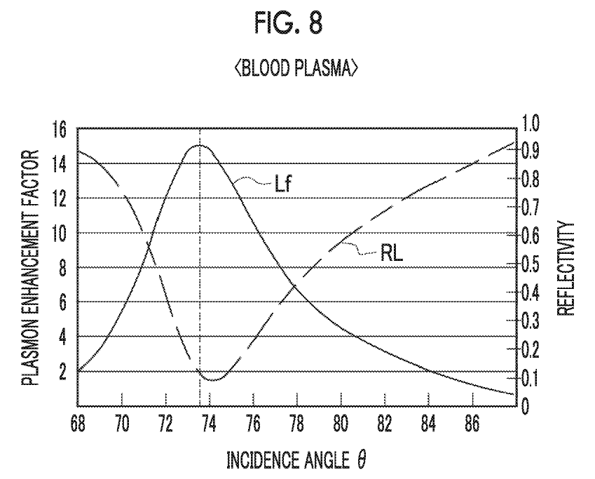

FIG. 8 shows a relationship between the incidence angle θ and each of a plasmon enhancement factor of the fluorescence Lf and a reflectivity of reflected light RL of the excitation light Le in a case where blood plasma is used as the specimen. The profile of FIG. 8 is an example in a case where a wavelength of the excitation light Le is 658 nm, a thickness of the metal film 18 is 36 nm, a material of the metal film 18 is gold, and a material of the prism 11A is polymethyl methacrylate (PMMA). Here, the plasmon enhancement factor is an indicator indicating how many times the light amount of the enhanced fluorescence Lf is with respect to a reference value, which is the light amount of the fluorescence Lf in a case where the enhancement is not performed. Since the plasmon enhancement factor is in a proportional relationship to the light amount of the fluorescence Lf detected by the fluorescence detection unit 32, in FIG. 8, even in a case where the vertical axis is set as the light amount of the fluorescence Lf, the relationship between the light amount of the fluorescence Lf and the incidence angle θ has the same profile.

In FIG. 8, the incidence angle θ at which the plasmon enhancement factor of the fluorescence Lf shows a peak value and is maximized is specified as the resonance angle. In the example shown in FIG. 8, the resonance angle is 73.6 degrees. Since the excitation light Le consumes energy for the plasmon enhancement, the reflected light RL of the excitation light Le is greatly attenuated near the resonance angle, and the reflectivity shows a minimum value, unlike the plasmon enhancement factor of the fluorescence Lf. By the incidence angle adjustment, the resonance angle at which the plasmon enhancement factor of the fluorescence Lf shows the maximum value as shown in FIG. 8 is specified.

FIG. 9 shows a state in which the specimen solution SL is injected into the flow path 15 to bring the specimen solution SL into contact with the reaction region 16. In the measurement device 100, the control unit 40 operates the pump 22 in a state in which the nozzle tip NC is inserted into the injection port 12 even after the specimen solution SL is injected into the flow path 15. The control unit 40 feeds the specimen solution SL in the flow path 15 by operating the pump 22. As described above, in a case of feeding the specimen solution SL, a pump may be connected to the discharge port 13 side, and suction may be performed in synchronization with the pump 22. In addition, a suction pump may be provided only on the discharge port 13 side instead of the pump 22, and the feeding of liquid may be performed by the suction operation of the suction pump.

Here, the liquid feeding direction is a direction in which the specimen solution SL flows from the injection port 12 side to the discharge port 13 side in the flow path 15.

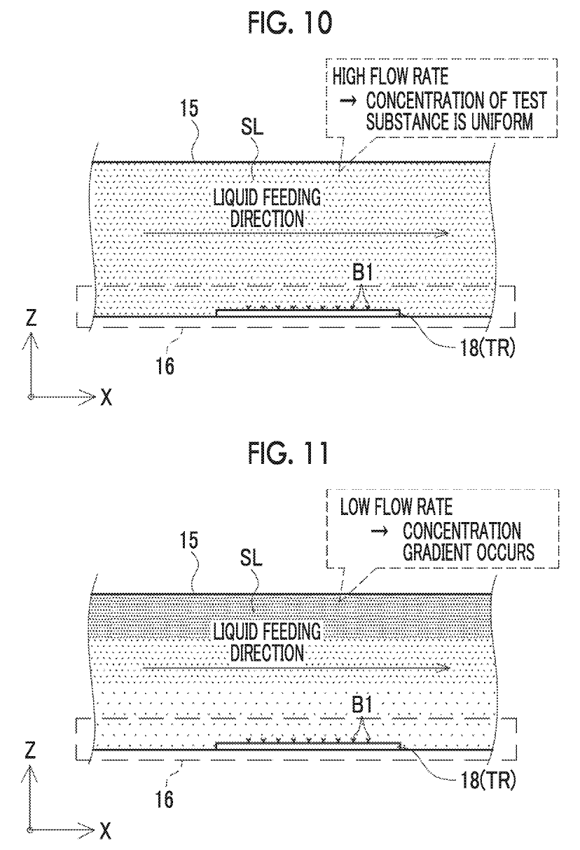

FIGS. 10 and 11 show a state in which the concentration distribution of the test substance A in the specimen solution SL changes depending on the flow rate of the specimen solution SL flowing in the flow path 15. FIG. 10 shows a case where the flow rate of the specimen solution SL is relatively high, and in this case, the concentration of the test substance A is relatively uniform in the specimen solution SL in the flow path 15. On the other hand, FIG. 11 shows a case where the flow rate of the specimen solution SL is relatively low, and in this case, the concentration of the test substance A is non-uniform in the specimen solution SL in the flow path 15, and a concentration gradient occurs. The concentration gradient is, as an example, a gradient in which the concentration is lower as the position is closer to the reaction region 16 in the flow path 15 and the concentration is higher as the position is farther from the reaction region 16. The concentration gradient increases as the flow rate is lower. That is, the concentration in the vicinity of the reaction region 16 decreases as the flow rate is lower.

In a case where the concentration gradient as shown in FIG. 11 occurs, the concentration of the test substance A is relatively reduced in the reaction region 16. Therefore, the measured value of the concentration of the test substance A measured by the measurement unit 30 is a value lower than the actual concentration of the test substance A in the specimen solution SL.

The reason why the concentration gradient as shown in FIG. 11 occurs is as follows. The test substance A present at a position close to the reaction region 16, that is, the test substance A present on a lower side in the Z direction, which is the up-down direction of the flow path 15 shown in FIG. 11, among the test substances A contained in the specimen solution SL flowing in the flow path 15, reacts with the first antibody B1. On the other hand, the test substance A present at a position far from the reaction region 16, that is, the test substance A present on the upper side of the flow path 15 does not react with the first antibody B1. Therefore, a difference in the concentration of the test substance A in the specimen solution SL occurs in the up-down direction of the flow path 15, which is the concentration gradient. In a case where the concentration gradient occurs, the test substance A spontaneously diffuses in a direction in which the concentration gradient disappears in the specimen solution SL, which is a diffusion movement. However, the speed of the diffusion movement is extremely slow compared to the reaction rate between the test substance A and the first antibody B1. Therefore, in a case where the flow rate of the specimen solution SL is lower than the target flow rate, the concentration gradient as shown in FIG. 11 occurs.

Such a concentration gradient is more remarkable as the flow rate is lower, and it typically tends to occur in a case where the measurement is performed in a state where the specimen solution SL is allowed to stand in a well plate or the like. The purpose of the liquid feeding method of performing the measurement while feeding the specimen solution SL is to make the concentration of the test substance A in the specimen solution SL uniform by the feeding of liquid, as shown in FIG. 10, and to improve the measurement accuracy as compared with a case of performing the measurement in a state where the specimen solution SL is allowed to stand in a well plate or the like.

However, due to the individual difference of the specimen solution SL, even in a case where the specimen solution SL is fed at the same liquid feeding pressure, the flow rate of the specimen solution SL may not reach the target flow rate, and in such a case, the concentration gradient may occur as shown in FIG. 11, and the measurement accuracy may be reduced.

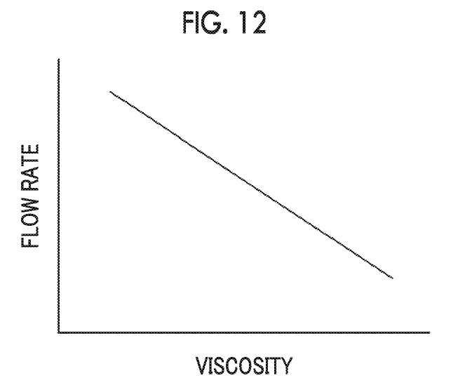

As shown in FIG. 12, the main factor of the decrease in the flow rate of the specimen solution SL is the viscosity of the specimen solution SL, and the flow rate is reduced as the viscosity is higher. The reason why the viscosity is high is the amount of an interfering substance that affects the flow rate of a protein or the like contained in the specimen, and the viscosity is higher as the amount of the interfering substance is larger. The amount of the interfering substance varies depending on the specimen solution SL.

Therefore, in the measurement device 100 of the present disclosure, the control unit 40 acquires flow rate-related information related to the flow rate of the specimen solution SL in the flow path 15, and executes post-processing related to reliability of a measurement result based on the flow rate-related information. That is, in a case where the viscosity of the specimen solution SL is high, even in a case where the liquid feeding pressure is the same, the flow rate is reduced, and the possibility that the concentration gradient of the test substance A as shown in FIG. 11 occurs in the flow path 15 is increased. In such a case, there is a concern about the reliability of the measurement result. Under such a premise, the control unit 40 executes the post-processing related to the reliability of the measurement result based on the flow rate-related information of the specimen solution SL.

In the present example, the flow rate-related information is an arrival time ΔT from the start of the feeding of the specimen solution SL to the arrival of the head BS of the specimen solution SL to the set position SP. That is, in a case where, as shown in the upper part of FIG. 13, a time at which the feeding of the specimen solution SL into the flow path 15 is started is defined as T1 and, as shown in the lower part of FIG. 13, a time at which the head BS of the specimen solution SL arrives to the set position SP is defined as T2, ΔT = T2 - T1. The control unit 40 measures a time from a time T1 at which the feeding of liquid is started, that is, a timing at which the pump 22 is started to operate, to a timing at which the head BS of the specimen solution SL arrives to the set position SP by the arrival detection unit 46, by the timer 40C. This measured time is the arrival time ΔT.

The set position SP is set on the downstream side of the reaction region 16, as an example. As described above, the timing at which the head BS of the specimen solution SL arrives to the set position SP is the switching timing from the primary liquid feeding to the secondary liquid feeding. The switching timing to the secondary liquid feeding is a timing at which the measurement by the measurement unit 30 is started. The measurement by the measurement unit 30 should be performed in a state where the reaction region 16 and the specimen solution SL are in contact with each other. Therefore, by setting the set position SP onto the downstream side of the reaction region 16, the measurement by the measurement unit 30 is started in a state where the specimen solution SL is in contact with the reaction region 16. The control unit 40 uses the arrival time ΔT to the set position SP as the flow rate-related information. Of course, in a case of simply using the arrival time ΔT to the set position SP as the flow rate-related information of the specimen solution SL, an arrival time to another position may be used instead of the arrival time ΔT to the set position SP.

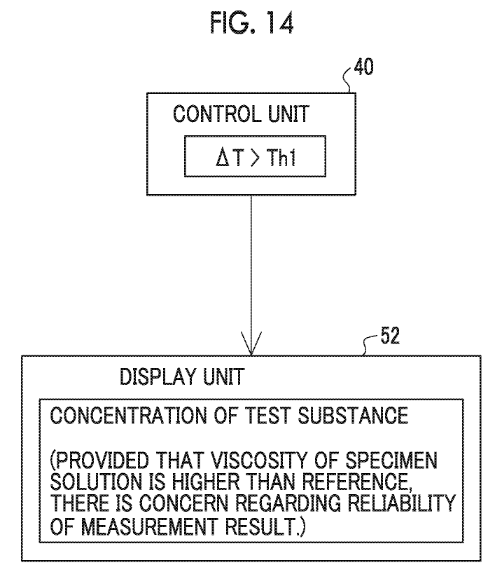

As shown in FIG. 14, in a case where the arrival time ΔT exceeds a first threshold value Th1 set in advance, the control unit 40 adds supplementary information related to reliability to the measurement result as the post-processing. The measurement result is the concentration of the test substance A. The first threshold value Th1 is set in consideration of, for example, the arrival time ΔT at which the influence on the reliability of the measurement result is not negligible.

As the supplementary information, for example, as shown in FIG. 14, a message that conveys a concern about the reliability of the measurement result, such as "the viscosity of the specimen solution is higher than the reference, and there is a concern about the reliability of the measurement result". The control unit 40 adds such supplementary information to the measurement result and outputs the measurement result as the post-processing. The post-processing of adding such supplementary information to the measurement result is, in other words, a warning related to the reliability of the measurement result.

The supplementary information may be a simple message indicating that the arrival time ΔT exceeds the first threshold value Th1. It is noted that, as the supplementary information, the message is shown as an example, but the supplementary information is not limited to the message, and may be a character or a mark indicating a concern about the reliability of the measurement result. Alternatively, a method of changing the color of the measurement result or changing the font may be used. In addition, as an output format of the supplementary information, the supplementary information may be displayed on the display unit 52, printed, or output as a voice.

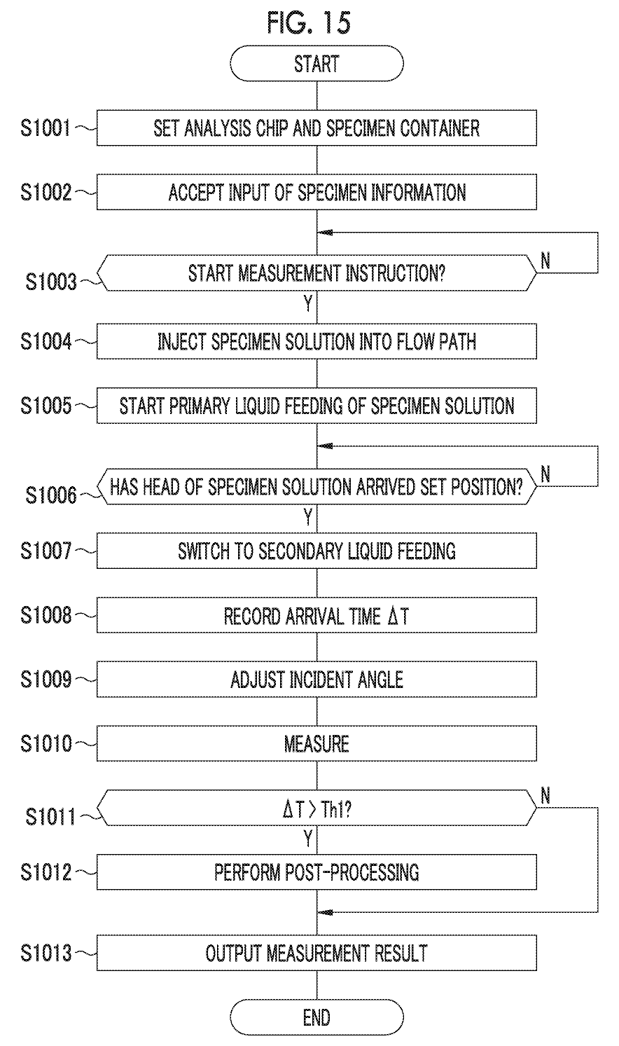

Hereinafter, the operation of the above-mentioned configuration will be described with reference to the flowchart shown in FIG. 15.

In a case of performing the measurement using the measurement device 100, first, Step (S1001) is executed, and the specimen container CB accommodating the analysis chip 10 and the specimen is set in the measurement device 100. The analysis chip 10 is mounted on the mounting portion 101.

Next, for example, specimen information such as patient information is input through the operation unit 51, and the control unit 40 receives the input specimen information (S1002). The control unit 40 waits for the input of the measurement start instruction from the operation unit 51 (S1003). In a case where the measurement start instruction is input (Y in S1003), the control unit 40 starts the operation of the specimen processing unit 20. As shown in FIG. 4, the specimen processing unit 20 suctions the specimen from the specimen container CB by using the nozzle tip NC. Then, as shown in FIG. 5, the suctioned specimen is injected from the nozzle tip NC to the reagent cell 14A, the fluorescent reagent containing the second antibody B2 and the fluorescent label F is mixed with the specimen, and the specimen solution SL is generated.

Then, the specimen processing unit 20 inserts a distal end of the nozzle tip NC into the injection port 12 of the analysis chip 10, and injects the generated specimen solution SL into the flow path 15 (S1004). After the specimen solution SL is injected into the flow path 15, the specimen processing unit 20 operates the pump 22 to start the primary liquid feeding of the specimen solution SL (S1005). The primary liquid feeding is performed at a higher liquid feeding pressure than the secondary liquid feeding. The liquid feeding pressure of the primary liquid feeding is constant regardless of the specimen solution SL. In a case where the primary liquid feeding is started, the control unit 40 operates the timer 40C to start the timing. Then, the control unit 40 monitors whether or not the head BS of the specimen solution SL has arrived to the set position SP based on the detection signal from the arrival detection unit 46 (S1006).

In a case where the head BS of the specimen solution SL arrives to the set position SP, the control unit 40 controls the pump 22 to reduce the liquid feeding pressure of the specimen solution SL and switches to the secondary liquid feeding (S1007). In addition, the control unit 40 records the arrival time ΔT measured by the timer 40C in the memory 40B (S1008).

In a case where the start of the secondary liquid feeding and the recording of the arrival time ΔT are ended, the control unit 40 adjusts the incidence angle of the excitation light irradiation unit 31 as a preparation for the measurement by the measurement unit 30 (S1009). The control unit 40 adjusts the incidence angle θ of the excitation light irradiation unit 31 to a reference angle set in advance. Then, the incidence angle θ is changed in a predetermined range in the positive direction and the negative direction with the reference angle as a reference in a state where the excitation light Le is irradiated from the excitation light irradiation unit 31. During this period, the fluorescence detection unit 32 detects the fluorescence corresponding to the reaction amount. The control unit 40 specifies the resonance angle at which the plasmon enhancement factor is maximized based on the fluorescence detection signal detected by the fluorescence detection unit 32.

After the incidence angle adjustment (S1009) is ended, the measurement is performed (S1010). The excitation light irradiation unit 31 irradiates the specimen with the excitation light Le at the specified resonance angle for a predetermined time, and during this period, the fluorescence detection unit 32 detects the fluorescence. Such fluorescence detection is performed for each of the first control region CR1, the test region TR, and the second control region CR2 in the reaction region 16 while moving the measurement unit 30.

In the measurement of S1010, the control unit 40 acquires the fluorescence detection signal detected by the fluorescence detection unit 32 in each of the first control region CR1, the test region TR, and the second control region CR2. Then, the reaction amount between the test substance A and the second antibody B2 is calculated, and the concentration of the test substance A in the specimen solution SL is calculated as the measurement result.

The control unit 40 determines whether or not the arrival time ΔT exceeds the first threshold value Th1 before outputting the measurement result (S1011). In a case where the arrival time ΔT exceeds the first threshold value Th1 (Y in S1011), the control unit 40 executes the post-processing (S1012). In the present example, the post-processing is to add the supplementary information related to the reliability to the measurement result as shown in FIG. 14. In S1013, the control unit 40 outputs the measurement result with the supplementary information added.

On the other hand, in a case where the arrival time ΔT is equal to or less than the first threshold value Th1 (N in S1011), the control unit 40 proceeds to S1013 without performing the post-processing, and outputs the measurement result.

As described above, the measurement device 100 according to the technology of the present disclosure includes the control unit 40 that is an example of the processor, and the control unit 40 acquires flow rate-related information related to the flow rate of the specimen solution SL in the flow path 15, and executes post-processing related to reliability of a measurement result based on the flow rate-related information. Therefore, an appropriate measure against reliability of a measurement result can be taken even in a case where there are individual differences in viscosity of specimen solutions SL.

In the above-described embodiment, the flow rate-related information is the arrival time ΔT until the head BS of the specimen solution SL arrives to the set position SP set in advance in the flow path 15 after the feeding of the specimen solution SL is started. The arrival time ΔT is a time in a case where the specimen solution SL is fed by a constant amount. Therefore, the arrival time ΔT is information in which a quantitative fluctuation factor of the specimen solution SL, such as a factor that takes time due to a large amount of the specimen solution SL to be fed, is eliminated, and is information that accurately reflects the individual difference in the viscosity of the specimen solution SL. By using the arrival time ΔT as the flow rate-related information, the control unit 40 can appropriately evaluate the reliability of the measurement result that is affected by the individual difference in the viscosity of the specimen solution SL, as compared with a case where the arrival time ΔT is not used.

In addition, the measurement device 100 according to the above-described embodiment measures the reaction by using the surface plasmon resonance. In such a measurement device 100, the technology of the present disclosure is particularly effective. This is because, as shown in FIGS. 7, 10, and 11, the surface plasmon resonance occurs in the vicinity of the reaction region 16. Therefore, in a case where the concentration of the test substance A in the vicinity of the reaction region 16 deviates from the actual concentration as in the concentration gradient shown in FIG. 11, the reliability of the measurement result using the surface plasmon resonance is greatly affected. Therefore, the technology of the present disclosure that realizes appropriate measures on the reliability of such a measurement result is particularly effective.

### Modification Example 1 of post-processing

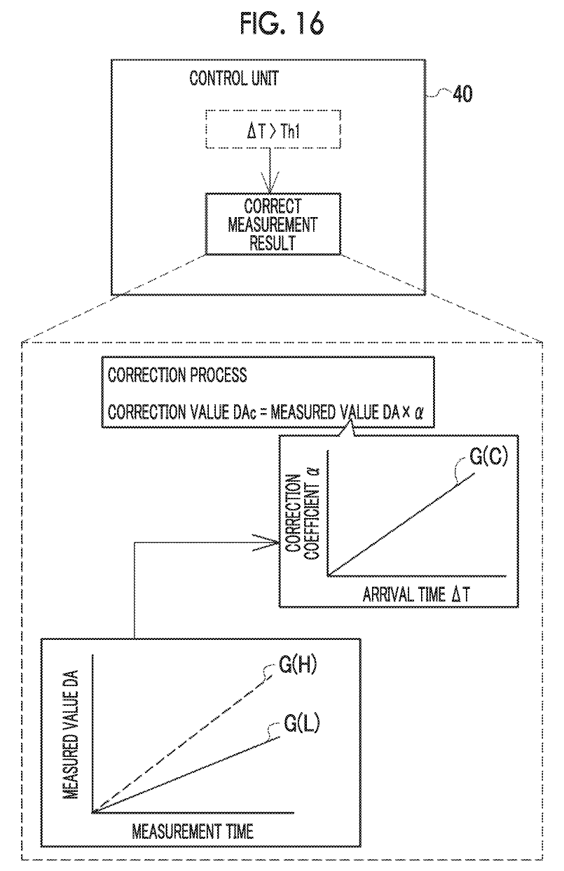

As shown in FIG. 16, as the post-processing related to the reliability of the measurement result, a correction processing of correcting the measurement result may be performed. In a case where the arrival time ΔT exceeds the first threshold value Th1, the control unit 40 corrects the measurement result. As an example of a method of determining the correction value, in a case where a measured value of the concentration of the test substance A by the measurement unit 30 is defined as DA, a correction coefficient is defined as α, and a correction value is defined as DAc, DAc = DA × α.

As described above, even in a case where the specimen solution SL having the same concentration of the test substance A is measured, the measured value DA is a value that is lower as the flow rate is lower due to the occurrence of the concentration gradient caused by the decrease in the flow rate of the specimen solution SL. A graph G(L) shown in FIG. 16 shows a relationship between the measurement time and the measured value DA in a case where the flow rate of the specimen solution SL is relatively slow, and a graph G(H) shows a relationship between the measurement time and the measured value DA in a case where the flow rate is relatively fast. The lower the flow rate of the specimen solution SL is, the lower the rising rate of the measured value DA with respect to the measurement time is. Therefore, the control unit 40 increases the correction coefficient α as the flow rate is lower, and corrects the correction value DAc to be larger. The correction coefficient α is set based on experimental results such as the graph G(L) and the graph G(H). Specifically, as shown in a graph G(C) of FIG. 16, the correction coefficient α is set to be larger as the arrival time ΔT, which is the flow rate-related information, is larger. The correlation between the correction coefficient α and the arrival time ΔT is stored in, for example, the memory 40B. The correlation may be in a form of table data or a form of a function.

Such a correction processing is a correction processing that ensures the reliability of the measurement result. The measurement device 100 can ensure the reliability of the correction result even in a case where the viscosity of the specimen solution SL fluctuates due to the individual difference by performing such a correction processing as the post-processing related to the reliability of the measurement result.

### Modification Example 2 of post-processing

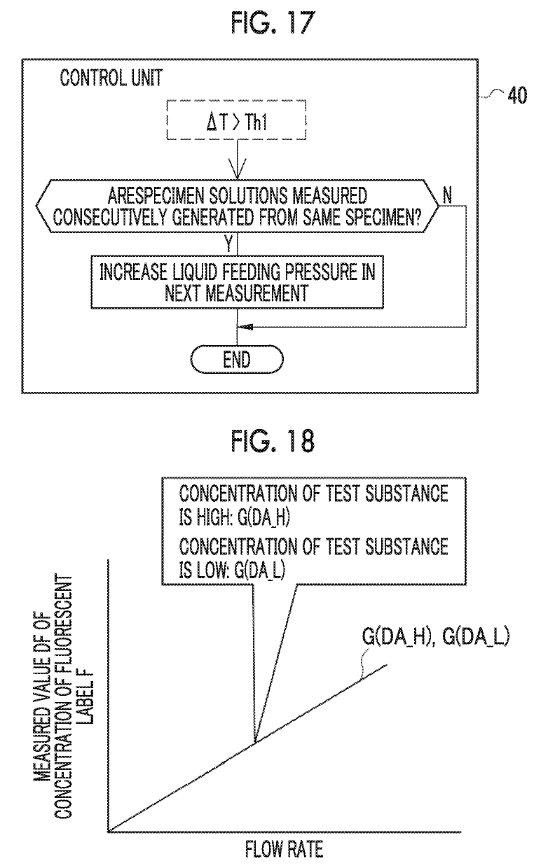

As shown in FIG. 17, and in a case where a plurality of the specimen solutions SL are continuously measured and the plurality of the specimen solutions SL that are consecutively measured are generated from the same specimen, the control unit 40 as an example of a processor is configured, in a case where the arrival time ΔT exceeds a predetermined first threshold value Th1 in the measurement of one specimen solution SL, to increase the liquid feeding pressure in a case of measuring another specimen solution SL that is consecutively measured as the post-processing.

In some cases, a plurality of measurements are consecutively performed with different reagents for a plurality of specimens. In such a case, a plurality of specimen solutions SL having the same specimen and different reagents are generated. In a case where the measurement is performed on one specimen solution SL among these, it is considered that the viscosity of the specimen contained in the specimen solution SL is high in a case where the arrival time ΔT exceeds the first threshold value Th1, and as a result, the flow rate is reduced. In such a case, the control unit 40 determines whether or not the specimen solution SL to be consecutively measured is generated from the same specimen, and in a case of the specimen solution SL generated from the same specimen, the control unit 40 increases the liquid feeding pressure in a case of measuring another specimen solution SL that is consecutively measured (that is, the liquid feeding pressure in a case of the next measurement) as the post-processing. As a result, the flow rate of the specimen solution SL increases, the concentration gradient of the test substance A in the flow path 15 is reduced, and the measurement accuracy of the next measurement is improved.

The process of increasing the liquid feeding pressure in the next measurement is a process of improving the reliability of the next measurement result. The measurement device 100 can improve the reliability of the next measurement result by performing the process of increasing the liquid feeding pressure in the next measurement as the post-processing related to the reliability of the measurement result.

### Second embodiment

The first embodiment is an aspect in which the arrival time ΔT is used as the flow rate-related information, but the second embodiment is an aspect in which the concentration of the label substance is used as the flow rate-related information. As described above, the specimen solution SL contains the fluorescent label F as the label substance that is bindable to the test substance A. In addition, in the analysis chip 10, the second control region CR2 is disposed as a capture region that captures the fluorescent label F regardless of whether or not the fluorescent label F is bonded to the test substance A on the downstream side of the reaction region 16 of the flow path 15. The control unit 40 uses a measured value DF (see FIG. 18) of the concentration of the fluorescent label F in the second control region CR2 as the flow rate-related information.

Even in a case where the concentration of the fluorescent label F contained in the specimen solution SL is the same, the measured value DF of the concentration of the fluorescent label F also changes in a case where the flow rate of the specimen solution SL changes. As shown in FIG. 18, a positive correlation is observed between the measured value DF and the flow rate. That is, the measured value DF is a value that is lower as the flow rate is lower, and the measured value DF is a value that is higher as the flow rate is higher. This is because, similarly to the test substance A described in FIGS. 10 and 11, the concentration gradient occurs due to the decrease in the flow rate.

In a graph G(DA_H) and a graph G(DA_L) shown in FIG. 18, the concentration of the fluorescent label F contained in the specimen solution SL is the same, but the graph G(DA_H) shows a case where the concentration of the test substance A is high, and the graph G(DA_L) shows a case where the concentration of the test substance A is low. That is, the graph G(DA_H) and the graph G(DA_L) are substantially the same, which indicates that the correlation between the flow rate and the measured value DF of the concentration of the fluorescent label F does not depend on the concentration of the test substance A.

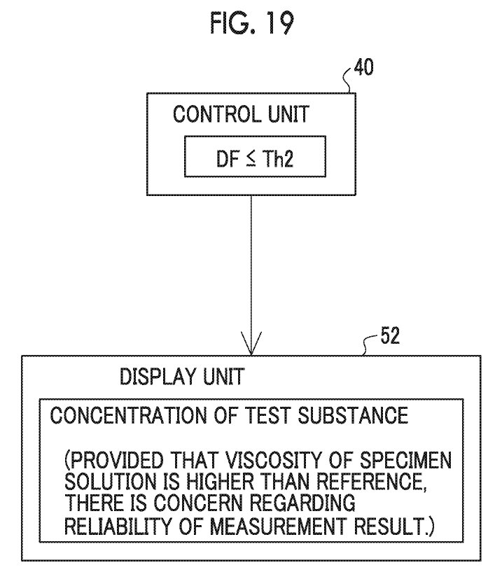

As shown in FIG. 19, the control unit 40 acquires the measured value DF of the concentration of the fluorescent label F based on the fluorescence detection signal of the second control region CR2 as the flow rate-related information, and compares the measured value DF with a second threshold value Th2 set in advance. The second threshold value Th2 is set in consideration of, for example, the measured value DF at which the influence on the reliability of the measurement result is not negligible. In a case where the measured value DF is equal to or less than the second threshold value Th2, the control unit 40 executes the post-processing related to the reliability of the measurement result similarly to the first embodiment. The example shown in FIG. 19 is the same process as the process of adding the supplementary information related to the reliability to the measurement result shown in FIG. 14.

As described above, as the flow rate-related information that is a reference in a case of executing the post-processing related to the reliability of the measurement result, the concentration of the fluorescent label F (an example of the label substance) in the second control region CR2 (an example of the capture region) can also be used. Also in the second embodiment, similarly to the first embodiment, an effect is obtained that it is possible to take an appropriate measures against reliability of measurement results even in a case where there are individual differences in viscosity of specimen solutions SL.

In addition, in the second embodiment, the example has been described in which the concentration of the fluorescent label F in the second control region CR2 is used as the flow rate-related information, but the flow rate of the specimen solution SL derived based on the concentration of the fluorescent label F may be used instead of using the concentration of the fluorescent label F itself. As shown in FIG. 18, since a positive correlation is observed between the measured value DF of the concentration of the fluorescent label F and the flow rate of the specimen solution SL, the flow rate can also be used as the flow rate-related information instead of the measured value DF.

In addition, as the post-processing in the second embodiment, the post-processing exemplified in the first embodiment can be appropriately combined in addition to the process of adding the supplementary information to the measurement result shown in FIG. 19. For example, the process of correcting the measurement result shown in FIG. 16 may be performed, or the process of increasing the liquid feeding pressure in the next measurement shown in FIG. 17 can be combined.

In addition, in each of the above-described embodiments, for example, a plurality of post-processings, such as the process of adding the supplementary information to the measurement result and the process of increasing the liquid feeding pressure in the next measurement, may be executed.

In addition, in the above-described embodiment, the example has been described in which the measurement by the measurement unit 30 is performed while the specimen solution SL is fed in one direction, but the measurement may be performed while the specimen solution SL is reciprocated in the flow path 15.

In addition, in the above-described embodiment, the example has been described in which the test substance A is an antigen, but the test substance A may be an antibody.

In addition, in the above-described embodiment, the measurement device using the surface plasmon resonance has been described as an example, but the present disclosure can also be applied to a measurement device that does not use the surface plasmon resonance. As a measurement device that does not use the surface plasmon resonance, for example, there is a measurement device that measures an adsorption amount of a substance on a sensor surface composed of a quartz crystal by using a quartz crystal microbalance method (QCM method). The quartz crystal is a structure in which a metal thin film is formed on both sides of a slice cut out from a crystal of quartz in a very thin plate shape. In a case where an alternating current electric field is applied to each metal thin film, the quartz crystal exhibits a property of vibrating at a certain resonance frequency. In a case where a minute amount of a substance is adsorbed on the metal thin film, the resonance frequency is reduced according to the mass. In the quartz crystal microbalance method, the adsorption amount of a substance adsorbed on the sensor surface is measured by using a correlation between the change in the mass of such a substance and the resonance frequency.

The following technical matters described in the following appendixes can be understood from the above description.

### Appendix 1

A measurement device that is configured to use an analysis chip having a flow path through which a specimen solution containing a target substance flows, the flow path being provided with a reaction region in which an antibody or an antigen that specifically reacts with the target substance is immobilized and with which the specimen solution comes into contact, and to measure a reaction of the antibody or the antigen with the target substance in the reaction region while feeding the specimen solution, the measurement device comprising: a processor, wherein the processor is configured to: acquires flow rate-related information related to a flow rate of the specimen solution in the flow channel; and executes post-processing related to reliability of a measurement result based on the flow rate-related information.

### Appendix 2

The measurement device according to appendix 1,

wherein the flow rate-related information is an arrival time until a head of the specimen solution arrives at a predetermined position in the flow path after initiation of the feeding of the specimen solution.

### Appendix 3

The measurement device according to appendix 2, wherein, in a case where the arrival time exceeds a predetermined first threshold value, the processor is configured to add supplementary information related to reliability to the measurement result as the post-processing.

### Appendix 4

The measurement device according to appendix 2 or 3, wherein, in a case where the arrival time exceeds a predetermined first threshold value, the processor is configured to correct the measurement result as the post-processing.

### Appendix 5

The measurement device according to any one of appendixes 2 to 4, wherein the liquid feeding pressure of the specimen solution is variable, and in a case where a plurality of the specimen solutions are continuously measured and the plurality of the specimen solutions that are consecutively measured are generated from the same specimen, the processor is configured, in a case where the arrival time exceeds a predetermined first threshold value in the measurement of one specimen solution, to increase the liquid feeding pressure in a case of measuring another specimen solution that is consecutively measured as the post-processing.

### Appendix 6

The measurement device according to appendix 1, wherein the specimen solution contains a label substance that is bindable to the test substance, a capture region that captures the label substance regardless of whether or not the label substance is bonded to the test substance is disposed on a downstream side of the reaction region of the flow path, and the flow rate-related information is a concentration of the label substance in the capture region or the flow rate of the specimen solution derived based on the concentration of the label substance.

### Appendix 7

The measurement device according to appendix 6, wherein, in a case where the concentration of the label substance or the flow rate is equal to or less than a predetermined second threshold value, the processor is configured to add supplementary information related to reliability to the measurement result as the post-processing.

### Appendix 8

The measurement device according to appendix 6 or 7, wherein, in a case where the concentration of the label substance or the flow rate is equal to or less than a predetermined second threshold value, the processor is configured to correct the measurement result as the post-processing.

### Appendix 9

The measurement device according to any one of appendixes 6 to 8, wherein the liquid feeding pressure of the specimen solution is variable, and in a case where a plurality of the specimen solutions are consecutively measured and the plurality of the specimen solutions that are continuously measured are generated from the same specimen, the processor is configured, in a case where the concentration of the label substance or the flow rate is equal to or less than a predetermined second threshold value in the measurement of one specimen solution, to increase the liquid feeding pressure in a case of measuring another specimen solution that is consecutively measured as the post-processing.

### Appendix 10

The measurement device according to any one of appendixes 1 to 9, wherein the reaction is measured by using surface plasmon resonance.

### Appendix 11

An operation method of a measurement device that is configured to use an analysis chip having a flow path through which a specimen solution containing a target substance flows, the flow path being provided with a reaction region in which an antibody or an antigen that specifically reacts with the target substance is immobilized and with which the specimen solution comes into contact, and to measure a reaction of the antibody or the antigen with the target substance in the reaction region while feeding the specimen solution, the operation method comprising executing, by a processor, processes including: a process of acquiring flow rate-related information related to a flow rate of the specimen solution in the flow channel; and a process of executing post-processing related to reliability of a measurement result based on the flow rate-related information.

### Appendix 12

An operation program of a measurement device that is configured to use an analysis chip having a flow path through which a specimen solution containing a target substance flows, the flow path being provided with a reaction region in which an antibody or an antigen that specifically reacts with the target substance is immobilized and with which the specimen solution comes into contact, and to measure a reaction of the antibody or the antigen with the target substance in the reaction region while feeding the specimen solution, the operation program causing a computer to execute processes including: a process of acquiring flow rate-related information related to a flow rate of the specimen solution in the flow channel; and a process of executing post-processing related to reliability of a measurement result based on the flow rate-related information.

Although the preferred embodiments of the present disclosure have been described above, the present disclosure is not limited to the above-described embodiments, and various configurations may of course be adopted, such as a combination of each embodiment and each modification example, without departing from the spirit of the present disclosure.

In addition, in the above-described embodiment, for example, as a hardware structure of the processor that executes various types of processing, such as the control unit 40, various processors shown below can be used. Various processors include a programmable logic device (PLD) that is capable of changing a circuit configuration after manufacturing, such as a field-programmable gate array (FPGA), and a dedicated electric circuit that is a processor having a circuit configuration dedicatedly designed for executing specific processing, such as an application specific integrated circuit (ASIC), in addition to a CPU that is a general-purpose processor configured to execute software (program) to function as various processing units.

Various types of processing described above may be executed by one of the various processors or may be executed by a combination of two or more processors (for example, a combination of a plurality of FPGAs or a CPU and an FPGA) of the same type or different types. A plurality of processing units may be configured by one processor. As an example in which the plurality of processing units are configured of one processor, there is a form in which a processor that realizes all functions of a system including the plurality of processing units by using one integrated circuit (IC) chip is used, such as a system on chip (SOC).

In this way, various processing units are configured by one or more of the above-described various processors as hardware structures.

Furthermore, the hardware structure of these various processors is, more specifically, an electric circuit (circuitry) in which circuit elements such as semiconductor elements are combined.

In addition to the operation program of the measurement device 100, the technology of the present disclosure extends to a computer readable storage medium (USB memory or digital versatile disc (DVD)-read only memory (ROM), or the like) that stores the operation program of the measurement device 100 in a non-transitory manner. In addition, an operation program according to the technology of the present disclosure can be provided as a program product. The program product includes products of every aspect for providing the program. For example, the program product includes a program provided through a network such as the Internet, and a non-transitory computer readable recording medium such as a CD-ROM or a DVD in which the program is stored.

Contents described and illustrated above are for detailed description of a part according to the present disclosed technology and are merely an example of the present disclosed technology. For example, description related to the above configurations, functions, actions, and effects is description related to examples of configurations, functions, actions, and effects of the parts according to the disclosed technology. Thus, unnecessary parts may be removed, new elements may be added, or the parts may be replaced with each other in the content of description and the content of illustration shown above without departing from the gist of the disclosed technology. In addition, in order to avoid complication and facilitate the understanding of a portion according to the present disclosed technology, regarding the contents described and illustrated above, description related to common technical knowledge or the like which does not need to be described to enable implementation of the present disclosed technology has been omitted.

In the present specification, “A and/or B” has the same meaning as “at least one of A or B”. That is, "A and/or B" may be only A, only B, or a combination of A and B. In addition, in the present specification, the same concept as in the case of “A and/or B” applies to a case where three or more matters are expressed together by “and/or”.

The disclosure of Japanese Patent Application No. 2023-112651 filed on July 7, 2023 is incorporated herein by reference in its entirety. In addition, all documents, patent applications, and technical standards described in the present specification are incorporated in the present specification by reference to the same extent as in a case where each document, patent application, and technical standard are specifically and individually noted to be incorporated by reference.

What is claimed is:

1. A measurement device that is configured to use an analysis chip having a flow path through which a specimen solution containing a target substance flows, the flow path being provided with a reaction region in which an antibody or an antigen that specifically reacts with the target substance is immobilized and with which the specimen solution comes into contact, and to measure a reaction of the antibody or the antigen with the target substance in the reaction region while feeding the specimen solution, the measurement device comprising: a processor,
wherein the processor is configured to:
acquires flow rate-related information related to a flow rate of the specimen solution in the flow path; and
executes post-processing related to reliability of a measurement result based on the flow rate-related information.

2. The measurement device according to claim 1,
wherein the flow rate-related information is an arrival time until a head of the specimen solution arrives at a predetermined position in the flow path after initiation of the feeding of the specimen solution.

3. The measurement device according to claim 2,
wherein, in a case where the arrival time exceeds a predetermined first threshold value, the processor is configured to add supplementary information related to reliability to the measurement result as the post-processing.

4. The measurement device according to claim 2,
wherein, in a case where the arrival time exceeds a predetermined first threshold value, the processor is configured to correct the measurement result as the post-processing.

5. The measurement device according to claim 2,
wherein the liquid feeding pressure of the specimen solution is variable, and
in a case where a plurality of the specimen solutions are continuously measured and the plurality of the specimen solutions that are consecutively measured are generated from the same specimen,
the processor is configured, in a case where the arrival time exceeds a predetermined first threshold value in the measurement of one specimen solution, to increase the liquid feeding pressure in a case of measuring another specimen solution that is consecutively measured as the post-processing.

6. The measurement device according to claim 1,
wherein the specimen solution contains a label substance that is bindable to the test substance,
a capture region that captures the label substance regardless of whether or not the label substance is bonded to the test substance is disposed on a downstream side of the reaction region of the flow path, and
the flow rate-related information is a concentration of the label substance in the capture region or the flow rate of the specimen solution derived based on the concentration of the label substance.

7. The measurement device according to claim 6,
wherein, in a case where the concentration of the label substance or the flow rate is equal to or less than a predetermined second threshold value, the processor is configured to add supplementary information related to reliability to the measurement result as the post-processing.

8. The measurement device according to claim 6,
wherein, in a case where the concentration of the label substance or the flow rate is equal to or less than a predetermined second threshold value, the processor is configured to correct the measurement result as the post-processing.

9. The measurement device according to claim 6,
wherein the liquid feeding pressure of the specimen solution is variable, and
in a case where a plurality of the specimen solutions are consecutively measured and the plurality of the specimen solutions that are continuously measured are generated from the same specimen,
the processor is configured, in a case where the concentration of the label substance or the flow rate is equal to or less than a predetermined second threshold value in the measurement of one specimen solution, to increase the liquid feeding pressure in a case of measuring another specimen solution that is consecutively measured as the post-processing.

10. The measurement device according to claim 1,
wherein the reaction is measured by using surface plasmon resonance.

11. An operation method of a measurement device that is configured to use an analysis chip having a flow path through which a specimen solution containing a target substance flows, the flow path being provided with a reaction region in which an antibody or an antigen that specifically reacts with the target substance is immobilized and with which the specimen solution comes into contact, and to measure a reaction of the antibody or the antigen with the target substance in the reaction region while feeding the specimen solution, the operation method comprising executing, by a processor, processes including:
a process of acquiring flow rate-related information related to a flow rate of the specimen solution in the flow path; and
a process of executing post-processing related to reliability of a measurement result based on the flow rate-related information.

12. A non-transitory computer-readable storage medium storing an operation program of a measurement device that is configured to use an analysis chip having a flow path through which a specimen solution containing a target substance flows, the flow path being provided with a reaction region in which an antibody or an antigen that specifically reacts with the target substance is immobilized and with which the specimen solution comes into contact, and to measure a reaction of the antibody or the antigen with the target substance in the reaction region while feeding the specimen solution, the operation program causing a computer to execute processes including:
a process of acquiring flow rate-related information related to a flow rate of the specimen solution in the flow path; and
a process of executing post-processing related to reliability of a measurement result based on the flow rate-related information.
